(19) United States
(12) Patent Application Publication
(10) Pub. No.: US 2018/0256534 A1
(43) Pub. Date: Sep. 13, 2018

(54) KETAMINE AND CYTOCHROME P 450 INHIBITOR COMBINATIONS

(71) Applicant: SEQUOIA PHARMACEUTICALS, INC., Potomac, MD (US)

(72) Inventor: John W. ERICKSON, Potomac, MD (US)

(21) Appl. No.: 15/912,131

(22) Filed: Mar. 5, 2018

Related U.S. Application Data
(63) Continuation of application No. PCT/US16/50442, filed on Sep. 6, 2016.
(60) Provisional application No. 62/214,837, filed on Sep. 4, 2015.

(51) Int. Cl. A61K 31/343; A61K 31/135; A61P 25/24

(52) U.S. Cl. CPC A61K 31/343; A61P 25/24; A61K 31/135

(57) ABSTRACT

Compositions and methods of treating depression infections are provided. More particularly, compositions including a combination of ketamine and a cytochrome p450 enzyme inhibitor are provided. Methods of using the compositions for treatment of depression, including treatment-resistant or treatment-refractory depression, are provided.

Compositions and methods of treating depression infections are provided. More particularly, compositions including a combination of ketamine and a cytochrome p450 enzyme inhibitor are provided. Methods of using the compositions for treatment of depression, including treatment-resistant or treatment-refractory depression, are provided.

This application is a continuation of International Application No. PCT/US16/50442, filed Sep. 6, 2016, which claims priority from U.S. Provisional Application No. 62/214,837, filed Sep. 4, 2015, the contents of each of which are hereby incorporated by reference in their entirety.

### FIELD OF THE TECHNOLOGY

The technology provides improved compositions and methods of treating depression, and particularly treatment-resistant or treatment-refractory depression. More specifically, the technology relates to compositions including a combination of ketamine (or ketamine active metabolites) and a cytochrome p450 enzyme inhibitor.

### BACKGROUND OF THE TECHNOLOGY

Major Depressive Disorder is defined as the presence of one of more major depressive episodes that are not better accounted for psychotic disorder or bipolar disorder. A major depressive episode is characterized by meeting five or more of the following criteria during the same 2 week period which represent a change in functioning and include depressed/sad mood, loss of interest and pleasure, indifference or apathy; and irritability and is usually associated with a change in sleep patterns, appetite and body weight, motor agitation or retardation, fatigue, impairment in concentration and decision making, feelings of shame or guilt, and thoughts of death or dying (Diagnostic and Statistical Manual of Mental Disorders, 4th Edition, American Psychiatric Association, 2004 (hereinafter “DSM IV”); Harrison's Principles of Internal Medicine, 2000). Symptoms of a depressive episode include depressed mood; markedly diminished interest or pleasure in all, or almost all, activities most of the day; weight loss when not dieting or weight gain, or decrease or increase in appetite nearly every day; insomnia or hypersomnia nearly every day; psychomotor agitation or retardation nearly every day; fatigue or loss of energy nearly every day; feelings of worthlessness or excessive or inappropriate guilt nearly every day; diminished ability to think or concentrate, or indecisiveness nearly every day; recurrent thoughts of death, recurrent suicidal ideation without a specific plan, or a suicide attempt or a specific plan for committing suicide. Further, the symptoms cause clinically significant distress or impairment in social, occupational, or other important areas of functioning. (DSM IV)

Current treatment options for unipolar depression include monotherapy or combination therapy with various classes of drugs including mono-amine oxidase inhibitors (MAOI), tricyclic antidepressants (TCA), serotonin specific reuptake inhibitors (SSRI), serotonin noradrenergic reuptake inhibitors (SNRI), and noradrenaline reuptake inhibitor (NRI). Examples include imipramine, amitriptyline, desipramine, nortriptyline, doxepin, protriptyline, trimipramine, maprotiline, amoxapine, trazodone, bupropion, chlomipramine, fluoxetine, citalopram, escitalopram, sertraline, paroxetine, tianeptine, nefazadone, venlafaxine, desvenlafaxine, duloxetine, reboxetine, mirtazapine, phenelzine, tranylcypromine, and/or moclobemide.

A substantial proportion of depressed patients that receive antidepressant therapy do not experience relief from depression symptoms. This group typifies level 1 treatment-resistant depression, that is, a failure to demonstrate an “adequate” response to an “adequate” treatment trial (that is, sufficient intensity of treatment for sufficient duration). Moreover, about approximately 30% of depressed patients remain partially or totally treatment-resistant to at least two antidepressant treatments including combination treatments.

Recently, ketamine (a racemic mixture of S- and R-enantiomers) and esketamine and arketamine (the S- and R-enantiomer of ketamine, respectively) have been shown to be efficacious in the treatment of depression (particularly in those who have not responded to other antidepressant treatment). Unless specifically defined otherwise, references to ketamine in this disclosure are to be understood to refer to racemic ketamine and/or its individual enantiomers.

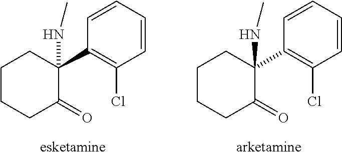 [SMILES: CN[C@@]1(c2ccccc2Cl)CCCCC1=O ; CN[C@]1(c2ccccc2Cl)CCCCC1=O]

In patients with major depressive disorders, ketamine has additionally been shown to produce a rapid antidepressant effect, acting within two hours. However, the usefulness of ketamine and its enantiomers has been limited by first pass metabolism, (leading to very short plasma half-life), and poor oral bioavailability. As a consequence, ketamine and its enantiomers must be given parenterally or intranasally. Both of these routes of administration are inconvenient and lead to poor patient compliance.

The poor bioavailability of orally administered ketamine is due in large part to its rapid metabolism by cytochrome P450 monooxygenase, leading to unfavorable pharmacokinetics. Therefore, oral administration of ketamine with an agent that inhibits metabolism by cytochrome P450 monooxygenase can improve the pharmacokinetics (i.e., increase half-life, increase the time to peak plasma concentration, increase blood levels) of the drug.

However, present methods of inhibiting cytochrome P450 enzymes are not wholly satisfactory because of toxicity issues, high cost, and other such factors. It is apparent, therefore, that new and improved agents and methods of inhibiting cytochrome P450-mediated degradation of ketamine are greatly to be desired. In particular, compositions and methods where the cytochrome p450 enzyme inhibitor can be co-administered with ketamine are highly desirable.

### SUMMARY OF THE TECHNOLOGY

The technology provides compositions and methods of treating depression, and particularly treatment-resistant or treatment-refractory depression. More particularly, the technology provides compositions including a combination of ketamine and cytochrome p450 enzyme inhibitors.

An advantage of the technology is that it provides improved combinations of ketamine and inhibitors of cytochrome P450 enzymes. Another advantage is that it provides a method of modifying or controlling the pharmacokinetic properties of ketamine. A further advantage is that it helps control the rate of metabolism or degradation of ketamine, thereby enhancing the bioavailability of ketamine. This enhances the efficacy of ketamine and can permit ketamine to be administered at a lower concentration or dosage, which reduces, for example, the chance of side effects.

More particularly, in one aspect, the technology provides a composition including a dose of ketamine effective for treating depression and a dose of a cytochrome inhibitor (“CYPI”) of the formula:

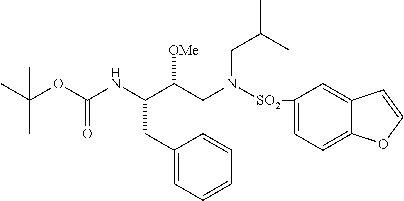 [SMILES: CO[C@H](CN(CC(C)C)S(=O)(=O)c1ccc2occc2c1)[C@H](Cc1ccccc1)NC(=O)OC(C)(C)C]

where the dose of the CYPI is effective to inhibit degradation and/or metabolism of ketamine when the composition is orally administered to a subject, particularly a human subject. The ketamine may be racemic ketamine or either enantiomer. Advantageously the ketamine is esketamine.

In other embodiments, the composition described above may be administered in combination with one or more antidepressants, and further in combination with one or more atypical antipsychotics.

In another aspect, the technology provides a method of treating depression or a depressive illness, including administering to a subject suffering from the disease an effective amount of the above compositions.

The details of one or more examples are set forth in the accompanying reaction schemes and description. Further features, aspects, and advantages of the technology will become apparent from the description, the schemes, and the claims.

### BRIEF DESCRIPTION OF THE DRAWING

FIG. 1 shows specific examples of cytochrome p450 inhibitors of the formula X-A-B-X′.

### DETAILED DESCRIPTION

The technology provides compositions and methods for treating depression, and especially for treating treatment-resistant or treatment-refractory depression. More particularly, the technology provides compositions including a combination of ketamine and cytochrome p450 enzyme inhibitors.

The technology provides methods of inhibiting cytochrome P450 (CYP) enzymes. The technology provides methods for enhancing the therapeutic effect of ketamine administered orally where the efficacy is compromised or eliminated due to degradation mediated by cytochrome P450. Upon administration, the compositions can provide serum concentrations of ketamine at a therapeutic level for a sustained period of time.

More particularly, in one aspect, the technology provides a composition including a dose of ketamine effective for treating depression and a dose of a cytochrome inhibitor (“CYPI”) of the formula I:

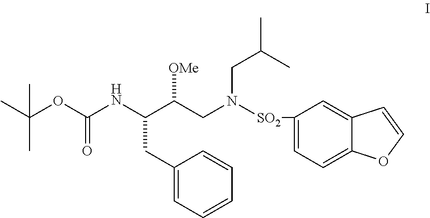 [SMILES: CO[C@H](CN(CC(C)C)S(=O)(=O)c1ccc2occc2c1)[C@H](Cc1ccccc1)NC(=O)OC(C)(C)C]

where the dose of the CYPI is effective to inhibit degradation and/or metabolism of ketamine when the composition is orally administered to a subject, particularly a human subject. The ketamine may be racemic ketamine or either enantiomer. Advantageously the ketamine is esketamine.

In other aspects the technology provides a composition including a dose of ketamine effective for treating depression and a dose of at least one cytochrome inhibitor represented by the formula X-A-B-X′, where:

X is a lipophilic group containing from 1 to 12 carbon atoms optionally containing from 1 to 3 heteroatoms independently selected from the group consisting of O, S, and N,

A is —OCON(R2)-, —S(O)ₙN(R2)-, —CON(R2)-, —COCO(NR2)-, —N(R2)CON(R2)-, —N(R2)S(O)ₙN(R2)-, N(R2)CO or —N(R2)COO—;

B is —(CG₁G₂)ₘ-, where m is 2-6 and where G₁ and G₂ are the same or different and where each G₁ and G₂ independently is selected from the group consisting of a bond, H, halo, haloalkyl, OR, optionally substituted alkyl, optionally substituted alkenyl, optionally substituted alkynyl, optionally substituted aryl, optionally substituted cycloalkyl, optionally substituted cycloalkylalkyl, optionally substituted aralkyl, optionally substituted heteroaryl, optionally substituted heteroaralkyl, and optionally substituted heterocycloalkyl where each optional substitution independently is selected from the group consisting of alkyl, halo, cyano, CF₃, OR, C₃-C₇ cycloalkyl, C₅-C₇ cycloalkenyl, R6, OR2, SR2, N(R2)₂, OR3, SR3, NR2R3, OR6, SR6, and NR2R6, and where G₁ and G₂, together with the atoms to which they are attached, optionally may form a 3-7-membered carbocyclic or heterocyclic ring containing up to three heteroatoms selected from the group consisting of N, S and O, and where the ring optionally may be substituted with up to 3 R7 moieties,

X′ is

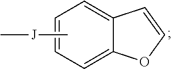

where J is selected from:

—N(D)-SOₙ—, —N(D)-COₙ—, —N(D)-(R8)_{q}—, —N(CO-D)-(R8)_{q}—, —N(SOₙ-D)-(R8)_{q}—, —SOₙ—N(D)-(R8)_{q}—, or —COₙ—N(D)-(R8)_{q}—,

where D is selected from hydrogen, alkyl, alkenyl, alkynyl, cycloalkyl, cycloalkylalkyl, heterocycloalkyl, heterocycloalkylalkyl, aryl, heteroaryl, heteroaralkyl or aralkyl, O-alkyl, O-cycloalkyl, O-cycloalkylalkyl, O-heterocycloalkyl, O-heterocycloalkylalkyl, O-heteroaralkyl O-aralkyl, N(R2)-alkyl, N(R2)-cycloalkyl, N(R2)-cycloalkylalkyl, N(R2)-heterocycloalkyl, N(R2)-heterocycloalkylalkyl, N(R2)-heteroaralkyl, N(R2)-aralkyl, wherein D optionally is substituted by alkyl, halo, nitro, cyano, O-alkyl, or S-alkyl;

where R is H, alkyl, haloalkyl, alkenyl, alkynyl, alkoxyalkyl, cycloalkyl, cycloalkylalkyl, heterocycloalkyl, heteroaryl, heterocycloalkylalkyl, aryl, aralkyl, and heteroaralkyl;

where each R2 is independently selected from the group consisting of H, C₁-C₁₂ alkyl, C₃-C₈ cycloalkyl, aryl, aralkyl, heteroaryl, heteroaralkyl, and heterocycloalkyl each further optionally substituted with one or more substituents selected from the group consisting of C₂-C₆ alkenyl, C₂-C₆ alkynyl, C₃-C₈ cycloalkyl, C₅-C₈ cycloalkenyl, heterocyclo; halo, OR, ROH, R-halo, NO₂, CN, COₙR, CON(R)₂, C(S)R, C(S)N(R)₂, SOₙN(R)₂, SR, SOₙR, N(R)₂, N(R)COₙR, NRS(O)ₙR, NRC[═N(R)]N(R)₂, N(R)N(R)COₙR, NRPOₙN(R)₂, NRPOₙOR, oxo, ═N—OR, ═N—N(R)₂, ═NR, ═NNRC(O)N(R)₂, ═NNRCOₙR, ═NNRS(O)ₙN(R)₂, and ═NNRS(O)ₙ(R);

or each R2 is independently selected from the group consisting of C₁-C₆ alkyl; substituted by aryl or heteroaryl; which groups optionally are substituted with one or more substituents selected from the group consisting of halo, OR, ROH, R-halo, NO₂, CN, COₙR, CON(R)₂, C(S)R, C(S)N(R)₂, SOₙN(R)₂, SR, SOₙR, N(R)₂, N(R)COₙR, NRS(O)ₙR, NRC[═N(R)]N(R)₂, N(R)N(R)COₙR, NRPOₙN(R)₂, NRPOₙOR;

R3 is C₂-C₆ alkenyl, C₂-C₆ alkynyl, C₃-C₈ cycloalkyl, C₅-C₈ cycloalkenyl, or heterocyclo; which groups optionally are substituted with one or more substituents selected from the group consisting of halo, OR2, R2-OH, R2-halo, NO₂, CN, COₙR2, C(O)N(R2)₂, C(O)N(R2)N(R2)₂, C(S)R2, C(S)N(R2)₂, S(O)ₙN(R2)₂, SR2, SOₙR2, N(R)₂, N(R2)COₙR2, NR2S(O)ₙR2, NR2C[═N(R2)]N(R2)₂, N(R2)N(R2)COₙR2, oxo, ═N—OR2, ═N—N(R2)₂, ═NR2, ═NNRC(O)N(R2)₂, ═NNR2C(O)ₙR2, ═NNR2S(O)ₙN(R2)₂, and ═NNR2S(O)ₙ(R2);

R6 is aryl or heteroaryl, where the aryl or heteroaryl optionally are substituted with one or more groups selected from the group consisting of aryl, heteroaryl, R2, R3, halo, OR2, R2OH, R2-halo, NO₂, CN, COₙR2, C(O)N(R2)₂, C(O)N(R2)N(R2)₂, C(S)R2, C(S)N(R2)₂, S(O)ₙN(R2)₂, SR2, SOₙR2, N(R)₂, N(R2)COₙR2, NR2S(O)ₙR2, NR2C[═N(R2)]N(R2)₂, N(R2)N(R2)COₙR2, OC(O)R2, OC(S)R2, OC(O)N(R2)₂, and OC(S)N(R2)₂;

R7 is H, oxo, C₁-C₁₂ alkyl; C₃-C₈ cycloalkyl, aryl, aralkyl, heteroaryl, heteroaralkyl, or heterocycloalkyl, each further optionally substituted with one or more substituents selected from the group consisting of C₂-C₆ alkenyl, C₂-C₆ alkynyl, C₃-C₈ cycloalkyl, C₅-C₈ cycloalkenyl, heterocyclo; halo, OR, ROH, R-halo, NO₂, CN, COₙR, CON(R)₂, C(S)R, C(S)N(R)₂, SOₙN(R)₂, SR, SOₙR, N(R)₂, N(R)COₙR, NRS(O)ₙR, NRC[═N(R)]N(R)₂, N(R)N(R)COₙR, NRPOₙN(R)₂, NRPOₙOR, oxo, ═N—OR, ═N—N(R)₂, ═NR, ═NNRC(O)N(R)₂, ═NNRCOₙR, ═NNRS(O)ₙN(R)₂, and ═NNRS(O)ₙ(R);

R8 is alkyl, haloalkyl, alkenyl, alkynyl, alkoxyalkyl, cycloalkyl, cycloalkylalkyl, heterocycloalkyl, heteroaryl, heterocycloalkylalkyl, aryl, aralkyl, and heteroaralkyl;

where n=1-2, and

where q=0-1.

In another aspect, X may be alkyl, alkenyl, alkynyl, alkoxyalkyl, cycloalkyl, cycloalkylalkyl, heterocycloalkyl, heteroaryl, heterocycloalkylalkyl, aryl, aralkyl, or heteroaralkyl; where X optionally is substituted with one or more substituents selected from the group consisting of C₂-C₆ alkenyl, C₂-C₆ alkynyl, C₃-C₈ cycloalkyl, C₅-C₈ cycloalkenyl, heterocyclo; halo, OR, ROH, R-halo, NO₂, CN, COₙR, CON(R)₂, C(S)R, C(S)N(R)₂, SOₙN(R)₂, SR, SOₙR, N(R)₂, N(R)COₙR, NRS(O)ₙR, NRC[═N(R)]N(R)₂, N(R)N(R)COₙR, NRPOₙN(R)₂, NRPOₙOR, oxo, ═N—OR, ═N—N(R)₂, ═NR, ═NNRC(O)N(R)₂, ═NNRCOₙR, ═NNRS(O)ₙN(R)₂, and ═NNRS(O)ₙ(R). In one embodiment, X may be selected from the group consisting of alkyl, cycloalkyl, aryl, aralkyl, heteroaryl, and heteroaralkyl. X optionally is substituted with one or more substituents selected from the group consisting of halo, OR, ROH, R-halo, CN, COₙR, CON(R)₂, SOₙN(R)₂, SR, SOₙR, N(R)₂, N(R)COₙR, NRS(O)ₙR, oxo, and ═N—OR.

In other aspects, G₁ and G₂ may be the same or different and independently are selected from the group consisting of a bond, H, OR, optionally substituted alkyl, optionally substituted aryl, optionally substituted cycloalkyl, optionally substituted cycloalkylalkyl, optionally substituted aralkyl, optionally substituted heteroaryl, and optionally substituted heteroaralkyl. In specific embodiments, G₁ and G₂ do not form a ring, or at least one G₁ and at least one G₂ form a ring. G₁ and G₂ may be different and, in certain embodiments, neither G₁ nor G₂ is OH.

In other aspects G₁ and G₂ are selected from the group consisting of H, O-alkyl, alkyl, optionally substituted aryl and optionally substituted aralkyl.

In the embodiments above, J may be

—N(D)-SOₙ—, —N(D)-COₙ—, —N(D)-(R8)_{q}—, —N(CO-D)-(R8)_{q}—, —N(SOₙ-D)-(R8)_{q}—, —SOₙ—N(D)-(R8)_{q}—, or —COₙ—N(D)-(R8)_{q}-.

In the embodiments above, D may be hydrogen, alkyl, alkenyl, alkynyl, cycloalkyl, cycloalkylalkyl, heterocycloalkyl, heterocycloalkylalkyl, aryl, heteroaryl, heteroaralkyl or aralkyl, O-alkyl, O-cycloalkyl, O-cycloalkylalkyl, O-heterocycloalkyl, O-heterocycloalkylalkyl, O-heteroaralkyl O-aralkyl, N(R2)-alkyl, N(R2)-cycloalkyl, N(R2)-cycloalkylalkyl, N(R2)-heterocycloalkyl, N(R2)-heterocycloalkylalkyl, or N(R2)-heteroaralkyl, N(R2)-aralkyl, where D optionally is substituted by alkyl, halo, nitro, cyano, O-alkyl, or S-alkyl.

In the compounds, when X is a 5-7 membered non-aromatic monocyclic heterocycle, optionally fused or bridged with one or more 3-7 membered non-aromatic monocyclic heterocycle to form a polycyclic system, where any of the heterocyclic ring systems contains one or more heteroatoms selected from O, N, S, and P, and

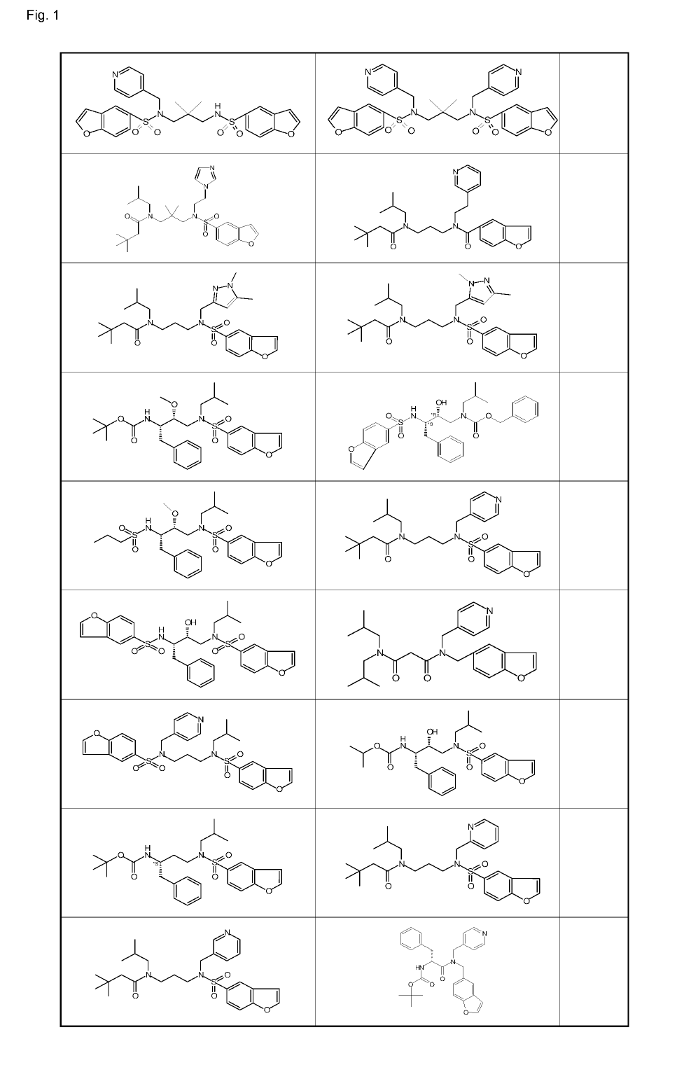

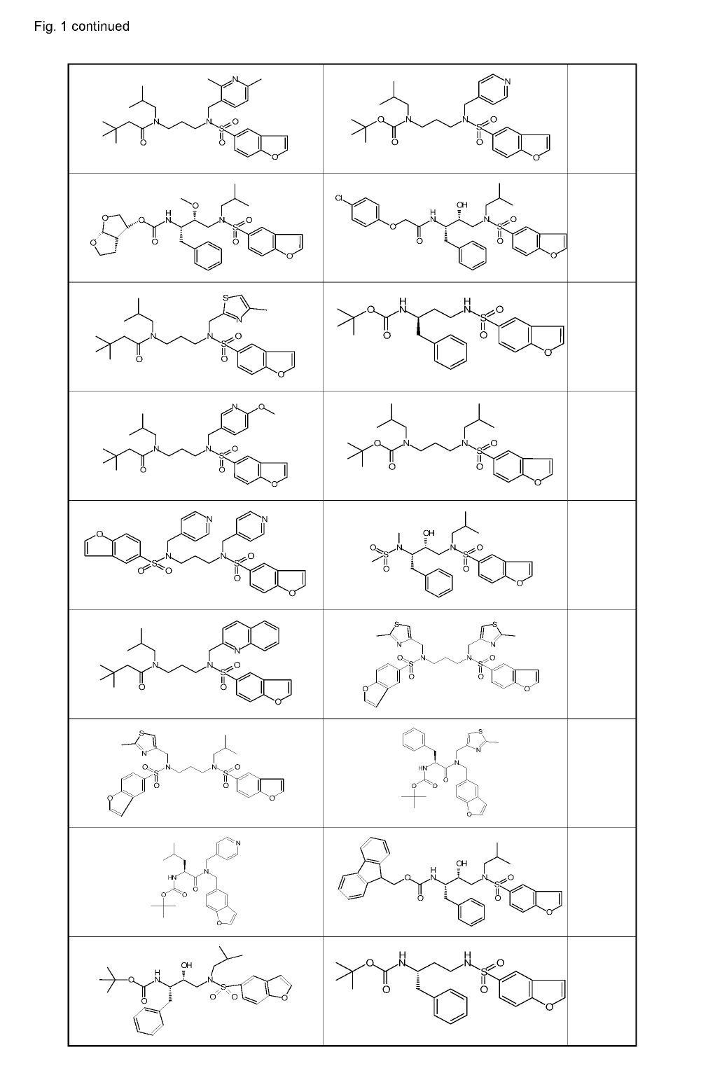

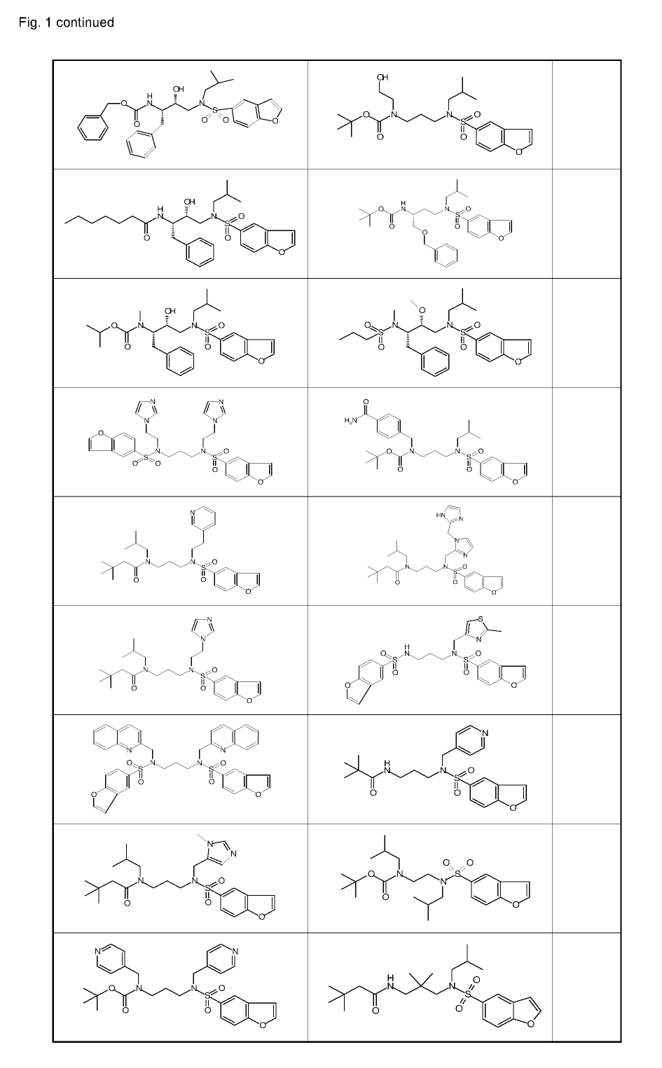

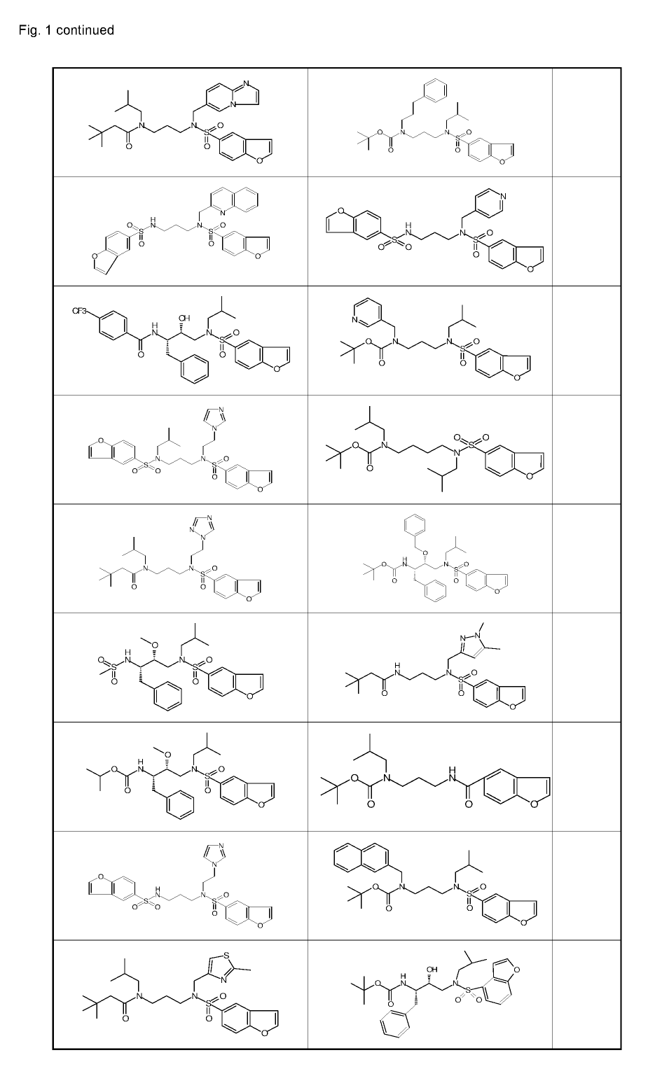

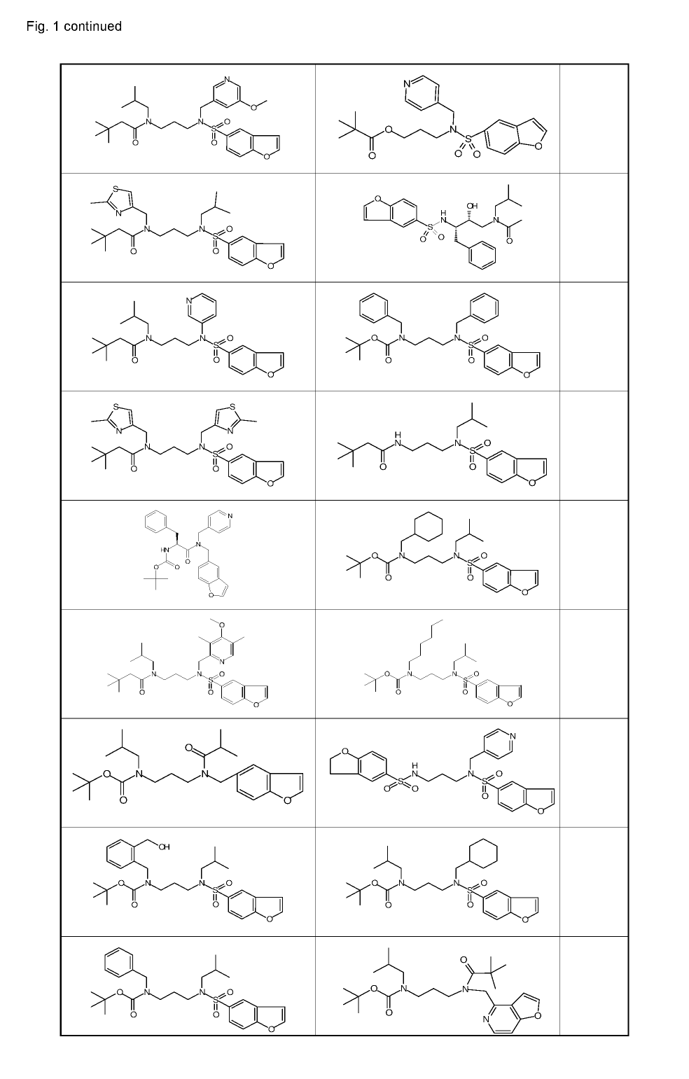

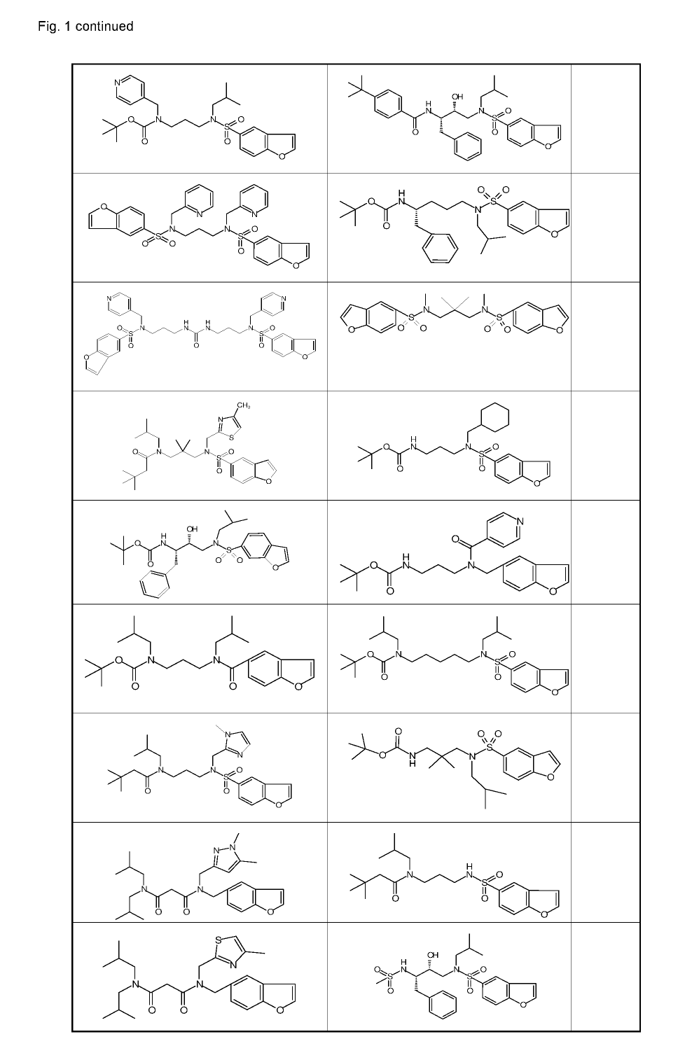

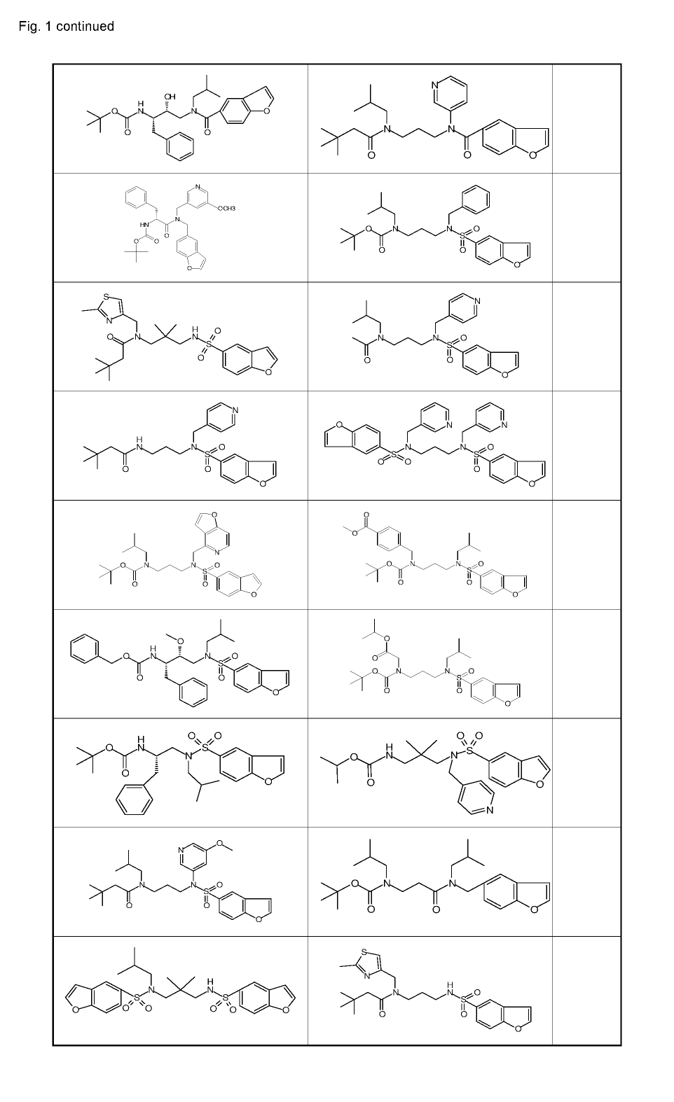

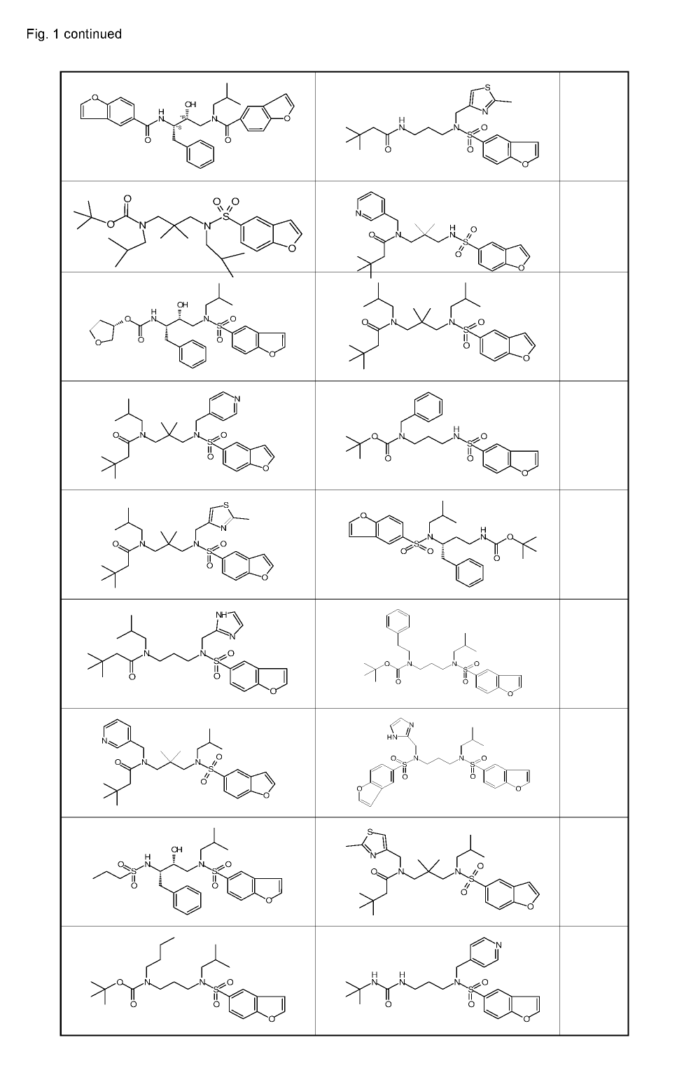

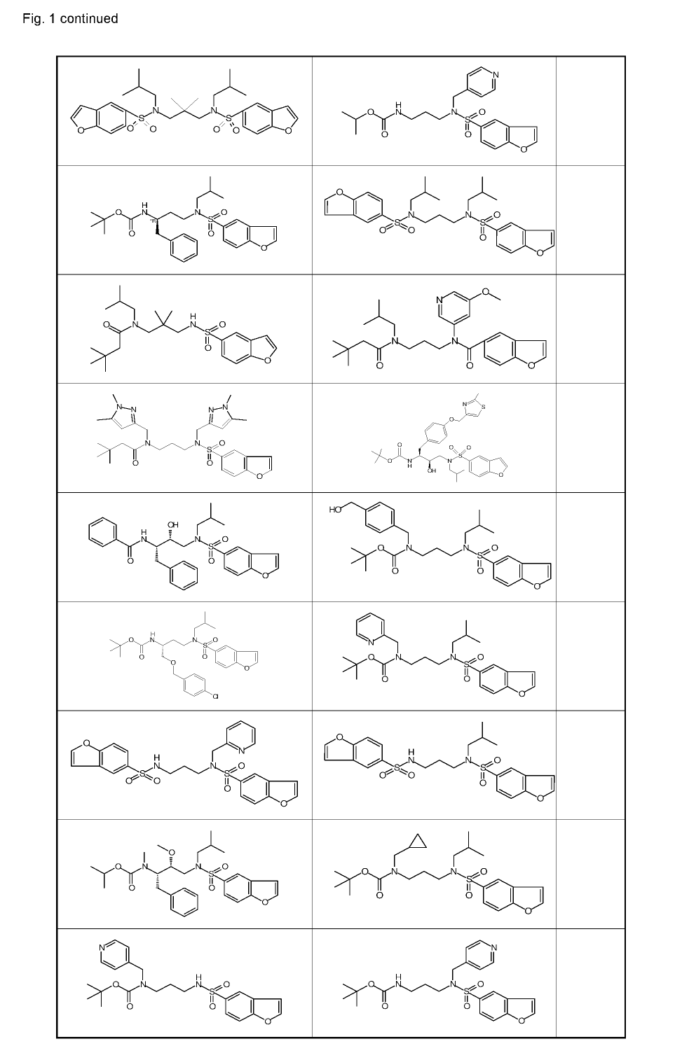

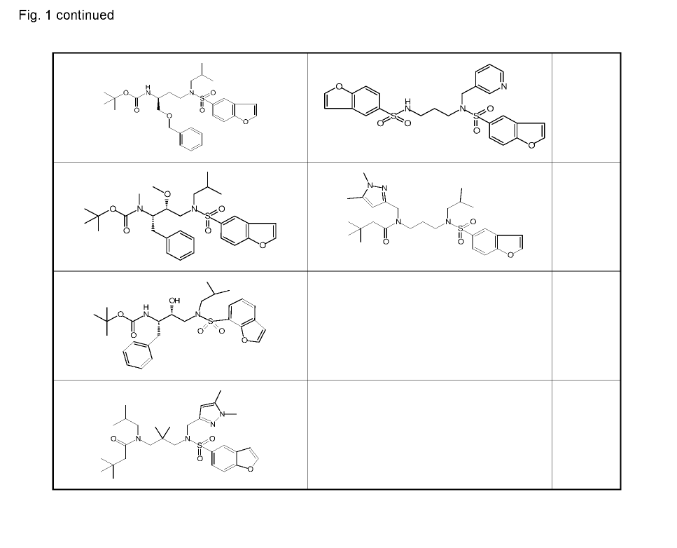

when B is

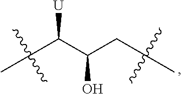

where U is selected from optionally substituted alkyl, optionally substituted alkenyl, optionally substituted alkynyl, optionally substituted aryl, optionally substituted cycloalkyl, or optionally substituted aralkyl, then J cannot be —N(D)-SOₙ— or —N(D)-COₙ. Specific examples of compounds of the formula X-A-B-X′ are shown in FIG. 1.

The term “pharmaceutically effective amount” as used herein refers to an amount of ketamine effective in treating depression. The term “treating” as used herein refers to the alleviation of symptoms of depression in a patient or the improvement of an ascertainable measurement of depression. As used herein, the term “patient” refers to a mammal, including a human.

Also included in the present application are one or more of the various polymorphs of the compounds. A crystalline compound disclosed in the present application may have a single or may have multiple polymorphs, and these polymorphs are intended to be included as compounds of the present application. Also, where a single polymorph is noted, the polymorph may change or interconvert to one or more different polymorphs, and such polymorph or polymorph mixtures are included in the present application.

Preparation and Assay of the Compounds

Ketamine, esketamine and arketamine are widely commercially available. The compound of the formula

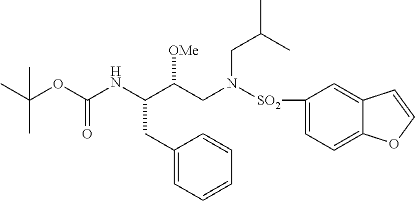 [SMILES: CO[C@H](CN(CC(C)C)S(=O)(=O)c1ccc2occc2c1)[C@H](Cc1ccccc1)NC(=O)OC(C)(C)C]

can be prepared by the methods described in U.S. Pat. No. 8,048,871, the contents of which are hereby incorporated by reference in their entirety. A specific synthesis of the CYPI compound is described below in Example 2. Reactions and processes for obtaining the compounds, particularly the formation of ester and amide linkages, may also be found in treatises and text, including, but not limited to, Advanced Organic Synthesis, 4th Edition, J. March, John Wiley & Sons, 1992 or Protective Groups in Organic Synthesis 3rd Edition, T. W. Green & P. G. M. Wuts, John Wiley & Sons, 1999, each of which is hereby incorporated by reference.

The starting materials and reagents used in preparing the CYPI compound are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Syntheses, Volumes 1-85 (John Wiley and Sons); Rodd's Chemistry of Carbon Compounds, Volumes 1-5 and Supplementals (Elsevier Science Publishers, 1989); Organic Reactions, Volumes 1-71 (John Wiley and Sons), Advanced Organic Synthesis, 4th Edition, J. March, John Wiley & Sons, 1992, and Larock's Comprehensive Organic Transformations (VCH Publishers Inc., 1989).

Protective groups, such as those described in Protective Groups in Organic Synthesis 3rd Edition, T. W. Green & P. G. M. Wuts, John Wiley & Sons, 1999 may be employed for a variety of purposes in the preparation of compounds encompassed by this disclosure. They may be employed to control the number or placement of substituents, or to protect functionalities that are otherwise unstable to reaction conditions employed for the introduction or modification of other substituents in a molecule. Where employed, such protective groups may be removed by suitable means. Alternatively, where the protective group is desirable in the product they may be introduced and not removed.

In certain embodiments, there is disclosed a method for improving the pharmacokinetics of ketamine (or a pharmaceutically acceptable salt thereof) by coadministering ketamine with the CYPI or a pharmaceutically acceptable salt thereof. When administered in combination, ketamine and the CYPI can be administered as a single composition.

### Methods of Administration

The compositions of this technology may be administered to a patient either as a single fixed-dose combination agent or in combination therapy with other antidepressant medications.

The combination may in some cases provide a synergistic effect, whereby depression and its associated symptoms may be prevented, substantially reduced, or eliminated completely.

The compounds of the technology can be administered in the form of pharmaceutically acceptable salts derived from inorganic or organic acids. Included among such acid salts, for example, are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate and undecanoate.

Other pharmaceutically acceptable salts include salts with an inorganic base, organic base, inorganic acid, organic acid, or basic or acidic amino acid. Inorganic bases which form pharmaceutically acceptable salts include alkali metals such as sodium or potassium, alkali earth metals such as calcium and magnesium, aluminum, and ammonia. Organic bases which form pharmaceutically acceptable salts include trimethylamine, triethylamine, pyridine, picoline, ethanolamine, diethanolamine, triethanolamine, dicyclohexylamine. Inorganic acids which form pharmaceutically acceptable salts include hydrochloric acid, hydroboric acid, nitric acid, sulfuric acid, and phosphoric acid. Organic acids appropriate to form salts include formic acid, acetic acid, trifluoroacetic acid, fumaric acid, oxalic acid, tartaric acid, maleic acid, citric acid, succinic acid, malic acid, methanesulfonic acid, benzenesulfonic acid, and p-toluenesulfonic acid. Basic amino acids used to form salts include arginine, lysine and ornithine. Acidic amino acids used to form salts include aspartic acid and glutamic acid.

The CYP inhibitory compounds described herein may be prepared and administered as a composition comprising a co-crystals with other compounds (co-crystal fomers). “Co-crystal” as used herein means a crystalline material comprised of two or more unique solids at room temperature, each containing distinctive physical characteristics, such as structure, melting point and heats of fusion. Co-crystals are described, for example, in U.S. Pub. No.: 20070026078 A1, which is incorporated by reference in its entirety. They are also described in, N. A. Meanwell, Annual Reports in Medicinal Chemistry, Volume 43, 2008 and D. P. McNamara, Pharmaceutical Research, Vol. 23, No. 8, 2006, each of which is incorporated by reference in its entirety.

The technology also contemplates compositions which can be administered orally or non-orally in the form of, for example, granules, powders, tablets, capsules, syrup, suppositories, injections, emulsions, elixirs, suspensions or solutions, by mixing these effective components, individually or simultaneously, with pharmaceutically acceptable carriers, excipients, binders, diluents or the like.

As a solid formulation for oral administration, the composition can be in the form of powders, granules, tablets, pills and capsules. In these cases, the compounds can be mixed with at least one additive, for example, sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. These formulations can contain, as in conventional cases, further additives, for example, an inactive diluent, a lubricant such as magnesium stearate, a preservative such as paraben or sorbic acid, an anti-oxidant such as ascorbic acid, tocopherol or cysteine, a disintegrator, a binder, a thickening agent, a buffer, a sweetener, a flavoring agent and a perfuming agent. Tablets and pills can further be prepared with enteric coating.

Examples of liquid preparations for oral administration include pharmaceutically acceptable emulsions, syrups, elixirs, suspensions and solutions, which can contain an inactive diluent, for example, water.

As used herein, “non-orally” includes subcutaneous injection, intravenous injection, intramuscular injection, intraperitoneal injection or instillation. Injectable preparations, for example sterile injectable aqueous suspensions or oil suspensions, can be prepared by known procedures in the fields concerned, using a suitable dispersant or wetting agent and suspending agent. The sterile injections can be, for example, a solution or a suspension, which is prepared with a non-toxic diluent administrable non-orally, such as an aqueous solution, or with a solvent employable for sterile injection. Examples of usable vehicles or acceptable solvents include water, Ringer's solution and an isotonic aqueous saline solution. Further, a sterile non-volatile oil can usually be employed as solvent or suspending agent. A non-volatile oil and a fatty acid can be used for this purpose, including natural or synthetic or semi-synthetic fatty acid oil or fatty acid, and natural or synthetic mono- or di- or tri-glycerides.

The pharmaceutical compositions can be formulated for nasal aerosol or inhalation and can be prepared as solutions in saline, and benzyl alcohol or other suitable preservatives, absorption promoters, fluorocarbons, or solubilizing or dispersing agents.

Rectal suppositories can be prepared by mixing the drug with a suitable vehicle, for example, cocoa butter and polyethylene glycol, which is in the solid state at ordinary temperatures, in the liquid state at temperatures in intestinal tubes and melts to release the drug.

In some embodiments, the pharmaceutical compositions can include α-, β-, or γ-cyclodextrins or their derivatives. In certain embodiments, co-solvents such as alcohols can improve the solubility and/or the stability of the compounds in pharmaceutical compositions. In the preparation of aqueous compositions, addition salts of the compounds can be suitable due to their increased water solubility.

Appropriate cyclodextrins are α-, β-, or γ-cyclodextrins (CDs) or ethers and mixed ethers thereof where one or more of the hydroxy groups of the anhydroglucose units of the cyclodextrin are substituted with C₁-C₆alkyl, such as methyl, ethyl or isopropyl, e.g. randomly methylated β-CD; hydroxy C₁₋₆alkyl, particularly hydroxyethyl, hydroxypropyl or hydroxybutyl; carboxy C₁-C₆alkyl, particularly carboxymethyl or carboxyethyl; C₁-C₆alkyl-carbonyl, particularly acetyl; C₁-C₆alkyloxycarbonylC₁-C₆alkyl or carboxyC₁-C₆alkyloxyC₁-C₆alkyl, particularly carboxymethoxypropyl or carboxyethoxypropyl; C₁-C₆alkylcarbonyloxyC₁-C₆alkyl, particularly 2-acetyloxypropyl. Especially noteworthy as complexants and/or solubilizers are β-CD, randomly methylated β-CD, 2,6-dimethyl-β-CD, 2-hydroxyethyl-β-CD, 2-hydroxyethyl-γ-CD, hydroxypropyl-γ-CD and (2-carboxymethoxy)propyl-β-CD, and in particular 2-hydroxypropyl-β-CD (2-HP-β-CD).

The term “mixed ether” denotes cyclodextrin derivatives where at least two cyclodextrin hydroxy groups are etherified with different groups such as, for example, hydroxypropyl and hydroxyethyl.

The compounds can be formulated in combination with a cyclodextrin or a derivative thereof as described in U.S. Pat. No. 5,707,975. Although the formulations described therein are with antifungal active ingredients, they are equally relevant for formulating compounds and compositions of the technology described herein (e.g., compositions comprising a compound of formula I and a compound of formula II). The formulations described therein are particularly suitable for oral administration and comprise an antifungal as active ingredient, a sufficient amount of a cyclodextrin or a derivative thereof as a solubilizer, an aqueous acidic medium as bulk liquid carrier and an alcoholic co-solvent that greatly simplifies the preparation of the composition. The formulations can also be rendered more palatable by adding pharmaceutically acceptable sweeteners and/or flavors.

Other convenient ways to enhance the solubility of the compounds of the technology in pharmaceutical compositions are described in WO 94/05263, WO 98/42318, EP-A-499,299 and WO 97/44014, all incorporated herein by reference.

In some embodiments, the compounds can be formulated in a pharmaceutical composition including a therapeutically effective amount of particles consisting of a solid dispersion including ketamine and the CYPI, and one or more pharmaceutically acceptable water-soluble polymers.

The term “solid dispersion” defines a system in a solid state including at least two components, where one component is dispersed more or less evenly throughout the other component or components. When the dispersion of the components is such that the system is chemically and physically uniform or homogenous throughout or consists of one phase as defined in thermodynamics, such a solid dispersion is referred to as “a solid solution”. Solid solutions are preferred physical systems because the components therein are usually readily bioavailable to the organisms to which they are administered.

The term “solid dispersion” also comprises dispersions which are less homogenous throughout than solid solutions. Such dispersions are not chemically and physically uniform throughout or comprise more than one phase.

The water-soluble polymer in the particles is conveniently a polymer that has an apparent viscosity of 1 to 100 mPa s when dissolved in a 2% aqueous solution at 20° C.

Preferred water-soluble polymers are hydroxypropyl methylcelluloses (HPMC). HPMC having a methoxy degree of substitution from about 0.8 to about 2.5 and a hydroxypropyl molar substitution from about 0.05 to about 3.0 are generally water soluble. Methoxy degree of substitution refers to the average number of methyl ether groups present per anhydroglucose unit of the cellulose molecule. Hydroxypropyl molar substitution refers to the average number of moles of propylene oxide which have reacted with each anhydroglucose unit of the cellulose molecule.

The particles as defined hereinabove can be prepared by first preparing a solid dispersion of the components, and then optionally grinding or milling that dispersion. Various techniques exist for preparing solid dispersions including melt-extrusion, spray-drying and solution-evaporation.

It can further be convenient to formulate the compounds in the form of nanoparticles which have a surface modifier adsorbed on the surface thereof in an amount sufficient to maintain an effective average particle size of less than 1000 nm. Useful surface modifiers are believed to include those which physically adhere to the surface of the antiretroviral agent but do not chemically bond to the antiretroviral agent.

Suitable surface modifiers can preferably be selected from known organic and inorganic pharmaceutical excipients. Such excipients include various polymers, low molecular weight oligomers, natural products and surfactants. Preferred surface modifiers include nonionic and anionic surfactants.

The compounds can also be incorporated in hydrophilic polymers and applied as a film over many small beads, thus yielding a composition with good bioavailability which can conveniently be manufactured and which is suitable for preparing pharmaceutical dosage forms for oral administration. The beads comprise a central, rounded or spherical core, a coating film of a hydrophilic polymer and an antiretroviral agent and a seal-coating polymer layer. Materials suitable for use as cores are pharmaceutically acceptable and have appropriate dimensions and firmness. Examples of such materials are polymers, inorganic substances, organic substances, saccharides and derivatives thereof. The route of administration can depend on the condition of the subject, co-medication and the like.

Dosages of the compounds and compositions described herein are dependent on age, body weight, general health conditions, sex, diet, dose interval, administration routes, excretion rate, combinations of drugs and conditions of the depression treated, while taking these and other necessary factors into consideration.

Generally, dosage levels of ketamine in the compositions are between about 5 μg/kg to about 10 mg/kg, preferably between about 0.5 mg/kg to about 5 mg/kg, 1 mg/kg to about 3 mg/kg, or a fixed dose between about 10-100 mg, or 20-75 mg, or 3-60 mg. The dosage of the CYPI in the combination can range about 10 μg to about 5000 mg, preferably between about 25 mg to about 1000 mg, or about 25 mg to about 250 mg. Typically, the pharmaceutical compositions of this technology will be orally administered from about 1 to about 3 times per day. Alternatively, sustained release formulations, may be employed. Sustained release formulations include, but not limited to, transdermal or iontophoretic patches, osmoitic devices, or sustained release tablets or suppositories that generally employ expandable or erodible polymer compositions. Such administrations can be used as a chronic or acute therapy.

The amount of active ingredient(s) that can be combined with the carrier materials to produce a single dosage form will vary depending upon the host treated and the particular mode of administration. A typical preparation will contain from about 5% to about 95% active compound (w/w). In some embodiments, such preparations contain from about 20% to about 80% active compound.

While these dosage ranges can be adjusted by a necessary unit base for dividing a daily dose, as described above, such doses are decided depending on the age, body weight, general health conditions, sex, diet of the patient when treated, dose intervals, administration routes, excretion rate, and combinations of drugs, while taking these and other necessary factors into consideration. For example, a typical preparation will contain from about 5% to about 95% active compound (w/w). Preferably, such preparations contain from about 10% to about 80% active compound. The desired unit dose of the composition of this technology is administered once or multiple times daily.

Advantageously, the compositions described herein are administered once a day and the dosages of ketamine and CYPI are sufficient to achieve a serum concentration of ketamine that is lower than about 50 ng/ml, which is the concentration at which psychotomimetic symptoms appear. Ketamine is also used as an analgesic, but the concentration of ketamine required to achieve relief from depression symptoms is lower than that required to achieve analgesia. Accordingly, the dosages of ketamine and CYPI in the composition are lower than the doses required to achieve ketamine-induced analgesia.

In some embodiments, the technology contemplates compositions and formulations including one or more of the compounds in combination with one or more other drugs that can be metabolized or degraded by CYP.

The compositions may also be administered with additional antidepressant compounds i.e. one or more pharmaceutical agents which can be used to treat depression. Suitable examples include, but are not limited to mono-amine oxidase inhibitors such as phenelzine, tranylcypromine, moclobemide, and the like; tricyclics such as imipramine, amitriptyline, desipramine, nortriptyline, doxepin, protriptyline, trimipramine, chlomipramine, amoxapine, and the like; tetracyclics such as maprotiline, and the like; non-cyclics such as nomifensine, and the like; triazolopyridines such as trazodone, and the like; serotonin reuptake inhibitors such as fluoxetine, sertraline, paroxetine, citalopram, citolapram, escitolapram, fluvoxamine, and the like; serotonin receptor antagonists such as nefazadone, and the like; serotonin noradrenergic reuptake inhibitors such as venlafaxine, milnacipran, desvenlafaxine, duloxetine and the like; noradrenergic and specific serotonergic agents such as mirtazapine, and the like; noradrenaline reuptake inhibitors such as reboxetine, edivoxetine and the like; atypical antidepressants such as bupropion, and the like; and lithium.

Therapeutically effective dosage levels and dosage regimens for antidepressants such as those described above may be readily determined by one of ordinary skill in the art. For example, therapeutic dosage amounts and regimens for pharmaceutical agents approved for sale are publicly available, for example as listed on packaging labels, in standard dosage guidelines, and in standard references.

The term “treatment-refractory or treatment-resistant depression” as used herein means a major depressive disorder that fails to respond to adequate courses of at least two antidepressants. Methods of determining whether a patient fails to respond to antidepressants are well known in the art.

Unless otherwise noted, the terms “treating,” “treatment” and the like, as used herein, include the management and care of a subject or patient, typically a human, for combating depression and include administration of a ketamine/CYPI fixed-dose combination as described herein to prevent the onset of the symptoms or complications, alleviate the symptoms or complications, or eliminate depression.

Unless otherwise noted, “prevention” of depression includes (a) reduction in the frequency of one or more symptoms of depression; (b) reduction in the severity of one or more symptoms of depression; (c) the delay or avoidance of the development of additional symptoms of depression; and/or (d) delay or avoidance of the development of depression.

The term “therapeutically effective amount” as used herein, means that amount of active compound or pharmaceutical agent that elicits the biological or medicinal response in a tissue system, animal or human that is being, including alleviation of the symptoms of depression.

Ketamine and the CYPI may be co-administered simultaneously, sequentially, separately or in a single pharmaceutical composition. Where the compounds are administered separately, the number of dosages of each compound given per day, may not necessarily be the same, e.g. where one compound may have a greater duration of activity, and will therefore, be administered less frequently. Further, the compounds may be administered via the same or different routes of administration, and at the same or different times during the course of the therapy, concurrently in divided or single combination forms. Advantageously, ketamine and the CYPI are administered in a single composition.

The following examples illustrate further the technology but, of course, should not be construed in any way of limiting its scope.

### Examples

### Example 1: Assay of IC₅₀ for the CYPI: Determinations Using Dibenzylfluorescein Metabolism by Human Liver Microsomes

A microtiter plate based, fluorometric assay was used for the determination of the concentration of the CYPI that will decrease by half the maximal rate of dibenzylfluorescein, a CYP3A4 substrate, metabolism by human liver microsomes. The assay was run as described by Crespi et al. Anal. Biochem. 248:188-90 (1997).

The test compound was diluted in acetonitrile in wells of a polypropylene microtiter plate (Denville Scientific, Inc. Metuchen, N.J.). Three fold serial dilutions of the test compound were made from the first well into the next seven wells of a row. Two wells of each row were used for positive controls containing no test compound and two for negatives containing 500 μM Ritonavir in acetonitrile. Test compounds in acetonitrile (0.004 mL) were added to wells of a microtiter plate (Catalog No. 3598, Corning Costar, Cambridge, Mass.) containing a solution (0.096 mL) of 0.2 M KPO4 Buffer (pH 7.4) and a NADPH generating system (2.6 mM NADP, 6.6 mM glucose-6-phosphate, 3.3 mM MgCl2 and 0.8 Units/mL G6P dehydrogenase (BD/Gentest, Woburn, Mass.). The plates were incubated for 10 minutes at 37° C. prior to addition of 0.1 mL of pre-warmed 0.1 mg/mL human liver microsomes (Xeno Tech, LLC, Lenexa, Kans.) in 0.2 M KPO4 buffer containing 2 μM dibenzylfluorescein (BD/Gentest, Woburn, Mass.). The plates were incubated for 10 minutes at 37° C. and the reaction are stopped by the addition of 0.075 mL of 2N NaOH. Plates were incubated at 37° C. for 1 hours prior to determining the amount of fluorescence in each well with a fluorescent plate reader (Spectra Max Gemini XS, Molecular Devices) at excitation/emission wavelengths of 485 and 538 nm (25 nm), respectively. Data were exported and analyzed using GraFit® (Erithacus Software Ltd., Surrey, U.K.). The background corrected data is fit to a 2-parameter equation for the determination of the IC₅₀.

### Example 2: Synthetic Methods

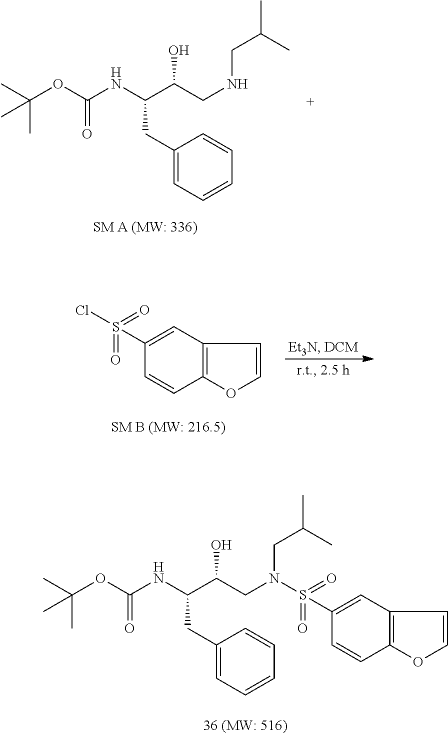

(1-Benzyl-2-hydroxy-3-isobutylamine-propyl)-carbamic acid tert-butyl ester (SM A, 10.08 g, 30 mmol, 1.0 equiv.) and 1-benzofuran-5-sulfonyl chloride (SM B, 9.74 g, 45 mmol, 1.5 equiv.) were dissolved in dichloromethane (100 mL). To the solution was added triethylamine (8.36 mL, 60 mmol, 2.0 equiv.) at room temperature. The mixture was stirred at the same temperature for 2.5 h, after which time the reaction was quenched through the addition of 0.5 N hydrochloric acid aqueous solution (50 mL). The phases were separated and then the organic layer was sequentially washed with 5% sodium bicarbonate (50 mL) and water (50 mL). The final organic solution was dried over anhydrous sodium sulfate and concentrated in vacuo. The residue was purified by recrystallization from ethyl acetate/hexane (30/90, v/v) to afford a white solid, 13.09 g, m.p. 121.1-122.4° C. The filtrate was concentrated and the residue was purified on silica gel (0-50% ethyl acetate in hexane) to afford 1.13 g additional target compound. Yield 14.22 g (92%). MS 1055 (2MNa)⁺, 539 (MNa)⁺, 417 (M-BOC)⁺ and 575 (AcOM)⁻. Purity 97% (HPLC).

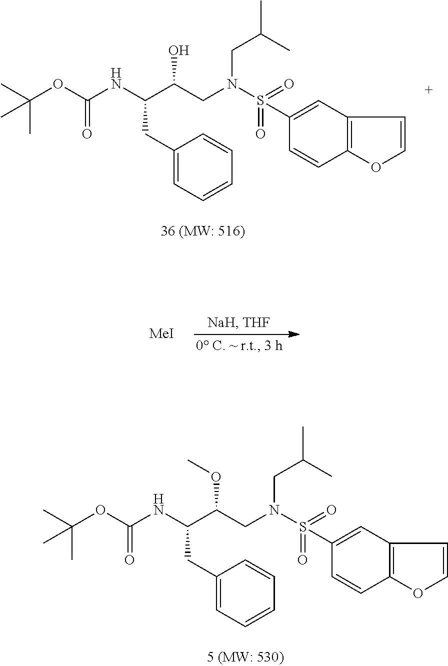 [SMILES: "MeI" ; CC(C)CN(C[C@@H](O)[C@H](Cc1ccccc1)NC(=O)OC(C)(C)C)S(=O)(=O)c1ccc2occc2c1 ; CO[C@H](CN(CC(C)C)S(=O)(=O)c1ccc2occc2c1)[C@H](Cc1ccccc1)NC(=O)OC(C)(C)C]

A 250 mL three-neck round-bottom flask was equipped with a magnetic stirbar, an argon inlet adapter and an air outlet adapter connected to a bubbler. The flask was charged with compound 36 (12.38 g, 24 mmol, 1.0 equiv.), anhydrous THF (96 mL), and methyl iodide (3.0 mL, 48 mmol, 2.0 equiv.) under argon. The mixture was cooled to 0° C. and treated with sodium hydride (1.92 g, 48 mmol, 2.0 equiv.) in portions. The resulting suspension was stirred for 3 h while the reaction was allowed to return to ambient temperature. Then 100 ml of water was added. The clear solution was concentrated in vacuo to remove the most of THF and was then extracted with ethyl acetate three times. The combined organic phase was washed with 0.5 N hydrochloric acid (50 mL), 5% sodium bicarbonate (50 mL), and brine (50 mL). It was then dried over anhydrous sodium sulfate and concentrated in vacuo to afford a yellow solid, which was purified by recrystallization from ethyl acetate/hexane (20/80, v/v) to afford a nearly colorless solid (9.15 g, 72%). A second recrystallization (ethyl acetate/hexane, 15/60) afforded a white solid (7.92 g), m.p. 115.3-115.8° C. ¹H NMR (δ, CDCl₃): 8.22 (s, 1H), 7.78-7.91 (m, 2H), 7.70 (d, J=8.4 Hz, 1H), 7.22-7.45 (m, 5H), 6.99 (s, 1H), 4.50-4.71 (m, 1H), 3.96-4.14 (m, 1H), 3.63-3.77 (m, 1H), 3.51 (s, 4H), 2.59-3.29 (m, 5H), 2.00-2.18 (m, 1H), 1.40 (s, 9H), 1.06 (d, J=6.4 Hz, 3H), 0.96 (d, J=6.4 Hz, 3H). MS 1083 (2MNa)⁺, 553 (MNa)⁺, 431 (M-BOC)⁺ and 589 (AcOM)⁻. Purity 96% (HPLC).

### Example 3: Efficacy of the Combination of Ketamine and the CYPI—(Prophetic Example)

The ability of the combination of ketamine and the CYPI to treat treatment-refractory or treatment-resistant depression is evaluated via a suitably designed clinical study. The study is a double-blind, double-randomization, placebo-controlled, multiple dose titration study in 30 adult subjects with treatment-resistant depression (TRD). The study consists of 3 phases: a screening phase of up to 2 weeks, a 7-day double-blind treatment phase (Day 1 to Day 7), and a 4-week post-treatment (follow up) phase.

Screening Phase: All subjects undergo a screening period of approximately 2 weeks, which provides adequate time to assess their eligibility per inclusion/exclusion criteria for the study.

Treatment Phase: On Day 1 of the treatment phase, a cohort of 30 adult subjects with TRD are randomized to one of three treatment groups (Group 1: composition containing 150 mg CYPI and 30 mg ketamine, Group 2: 150 mg CYPI and 15 mg ketamine, or Group 3: 150 mg CYPI and placebo). If the 30 mg ketamine dose is not well tolerated, the dose may be reduced to 20 mg. The compositions are administered daily.

Subjects who have a reduction in MADRS total score of >50% versus baseline on Day 2, 3, or 4 (prior to dosing) are considered responders. For subjects who are not responders after 3 days of treatment, treatment on Day 4 is selected as follows: (a) If the subject was treated with Placebo: the subject is then re-randomized to daily treatment with a 30 mg or 15 mg ketamine dose on Day 4; (b) if the subject was treated with 15 mg ketamine: the subject is assigned to treatment with 30 mg ketamine from Day 4 on; (c) If the subject was treated with 30 mg ketamine: the subject is then assigned to continue treatment with 30 mg ketamine.

Follow-Up: One week (7 days) after the end of the double-blind treatment phase (Day 14), subjects are assessed again. Additional assessments conducted 3 (i.e., Day 10), 10 (i.e., Day 17), 14 (i.e., Day 21), 21 (i.e., Day 28), and 28 (i.e., Day 35) days after the end of the double-blind treatment phase. The interval between the first and last dose of study medication is 3 days. The total study duration for each subject is a maximum of 7 weeks. The end of study is defined as the date of the last study assessment of the last subject in the trial.

Clinical Assessment of Efficacy: The primary efficacy evaluation is the Montgomery-Asberg Depression Rating Scale (MADRS) total score including modified versions for 24-hours and 2-hours recall. Secondary evaluations include evaluation of (a) MDD symptoms using the Quick Inventory of Depressive Symptomatology-Self Report-16-item (7-days recall) with modified 14-item (24-hours recall) and 10-item (2-hours recall) versions; (b) the severity of illness based on the Clinical Global Impression—Severity (CGI-S) and the global change in major depressive disorder (MDD) based on the Clinical Global Impression—Improvement (CGI-I); (c) the severity of illness based on subject's impression using the PGI-S; and (d) patient perspective of global change in MDD since start of study treatment, as measured by PG I-C.

Additional clinical evaluations include PK venous blood samples for measurement of ketamine and norketamine plasma concentrations, with a first PK sample on Day 1 (to evaluate the single-dose PK of ketamine) and an additional PK sample collected on Day 4 (to evaluate the maximum ketamine concentrations). Physical examination, body weight, vital signs, digital pulse oximetry, 12-lead ECG, continuous ECG monitoring, clinical laboratory tests (chemistry, hematology, urinalysis), and evaluation of adverse events are performed throughout the study to monitor subject safety. The collection of adverse events and recording of concomitant therapies is started after the informed consent has been signed and continues until the final follow up assessment. Other safety evaluations include the C-SSRS (to assess risk of suicide), BPRS (to assess severity of emergent psychotic symptoms), MGH-CPFQ (to assess cognitive and executive dysfunction) and the CADSS (to assess severity of emergent dissociative symptoms).

Results/Analysis: The primary endpoint is the change in the MADRS total score after each day of treatment. The primary comparison is between each ketamine/CYPI treatment group and the CYPI/placebo treatment group.

A mixed-effects model using repeated measures (MMRM) is performed on the change from baseline in MADRS total score up to Day 4. The model includes baseline score as covariate, and day, treatment, center and day-by-treatment interaction as fixed effects, and a random subject effect. Appropriate contrasts are used to determine the estimated differences between each ketamine dose and placebo. The contrast on Day 2 changes is of primary interest, and tested one-sidedly at the alpha level of 0.10.

Subjects who have a reduction in MADRS total score of >50% versus baseline on Day 2, 3, or 4 (prior to dosing) are considered responders. The response rate in each ketamine group are compared with placebo using the exact Mantel-Haenszel test stratified by center as a secondary analysis. Similar analyses are performed on secondary efficacy endpoints.

Variations, modifications, and other implementations of what is described herein will occur to those of ordinary skill in the art without departing from the spirit and the scope of the technology. Accordingly, the technology is not to be limited only to the preceding illustrative descriptions.

What is claimed is:

1. A composition comprising a therapeutically effective dose of ketamine, esketamine and/or arketamine and an effective dose of a CYPI compound of the formula

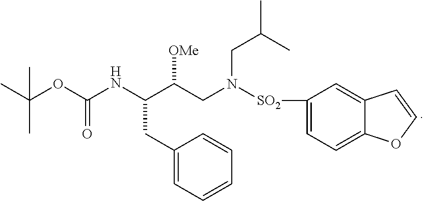 [SMILES: CO[C@H](CN(CC(C)C)S(=O)(=O)c1ccc2occc2c1)[C@H](Cc1ccccc1)NC(=O)OC(C)(C)C]

2. The composition according to claim 1 wherein said effective dose of ketamine is a dosage that is effective to treat depression.

3. The composition according to claim 2 wherein said depression is treatment-resistant or treatment-refractory depression.

4. The composition according to claim 1 wherein the dose of said CYPI compound is sufficient to inhibit degradation of ketamine, esketamine and/or arketamine in vivo such that a therapeutically effective serum concentration of ketamine, esketamine and/or arketamine is achieved after oral administration of said composition to a human subject.

5. A composition comprising a therapeutically effective dose of esketamine and an effective dose of a CYPI compound of the formula

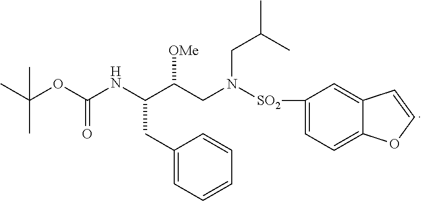 [SMILES: CO[C@H](CN(CC(C)C)S(=O)(=O)c1ccc2occc2c1)[C@H](Cc1ccccc1)NC(=O)OC(C)(C)C]

6. A method of treating depression comprising administering to a patient suffering from depression a composition according to claim 1.

7. A method of treating depression comprising administering to a patient suffering from depression a composition according to claim 5.

8. The method according to claim 5 wherein said depression is treatment-resistant or treatment-refractory depression.
